(19) United States
(12) Patent Application Publication
(10) Pub. No.: US 2026/0183154 A1
(43) Pub. Date: Jul. 2, 2026

(54) ABSORBENT ARTICLES HAVING REACTIVE GRAPHICS FOR PROVIDING CONTEXT-RELEVANT DIGITAL EXPERIENCES

(71) Applicant: KIMBERLY-CLARK WORLDWIDE, INC., Neenah, WI (US)

(72) Inventors: Jonathan D. BOULOS, Appleton, WI (US); Joseph P. FELL, Kaukauna, WI (US); Shawn J. SULLIVAN, Neenah, WI (US); Theodore T. TOWER, Neenah, WI (US)

(21) Appl. No.: 19/129,716

(22) PCT Filed: Dec. 23, 2022
(86) PCT No.: PCT/US2022/053947

§ 371 Date: May 14, 2025

(51) Int. Cl. A61F 13/42; A61F 13/49; A61F 13/84; A61L 15/56

(52) U.S. Cl. CPC A61F 13/42; A61F 13/49; A61L 15/56; A61F 2013/429; A61F 2013/8497

(57) ABSTRACT

An absorbent article includes an outer cover, an absorbent inner structure, and a reactive graphic visible on an exterior surface of the outer cover. The reactive graphic is formed of a reactive ink that, when exposed to an activation event, transitions from a first state to a second state. In the first state, the reactive graphic forms a first machine-readable code that includes a first digital content link and, in the second state, the reactive graphic forms a second machine readable code that includes a second digital content link. Generally, the second machine-readable code is different from the first machine-readable code and replaces the first machine-readable code when the reactive ink transitions from the first state to the second state, and the second digital content link is different from the first digital content link.

### BACKGROUND

Absorbent articles such as diapers, training pants, incontinence products, feminine hygiene products, and the like conventionally include a liquid permeable bodyside liner, a liquid impermeable outer cover, and an absorbent core. The absorbent core is typically located between the outer cover and the liner for taking in and retaining body fluids (e.g., urine) exuded by the wearer. The absorbent core can be made of, for example, superabsorbent particles or other absorbent materials. In some cases, absorbent articles may be adorned (e.g., on the liquid impermeable outer cover) with printed graphics, such as decorative images or patterns.

However, the use of absorbent articles is often accompanied by user-related challenges. Parents, for example, are continually challenged to motivate children wearing diapers to become potty-trained. Similarly, parents also seek to ensure children are comfortably clothed and, thus, seek to reduce the extent to which children are exposed to skin-contacting moisture and/or excessive heat. Accordingly, there remains a need for absorbent articles configured for providing user information and an enhanced user experience.

### SUMMARY

Some of the aspects of the present disclosure relate to an absorbent article including an outer cover having an interior surface and an exterior surface, an absorbent structure having a first surface positioned adjacent to the interior surface of the outer cover and a second surface that faces a wearer, the second surface being opposite of the first surface. Some aspects relate to an absorbent article including an outer cover having an interior surface and an exterior surface, an absorbent structure having a first surface positioned adjacent to the interior surface of the outer cover and a second surface that faces a wearer, the second surface being opposite of the first surface, and a reactive graphic visible at a first position on the exterior surface of the outer cover, the reactive graphic including a reactive ink that, when exposed to an activation event, transitions from a first state to a second state.

In some aspects, in addition, or in alternative to any preceding aspects, in the first state, the reactive graphic forms a first machine-readable code at the first position on the exterior surface of the outer cover, the first machine-readable code including a first digital content link.

In some aspects, in addition, or in alternative to any preceding aspects, in the second state, the reactive graphic forms a second machine-readable code at the first position on the exterior surface of the outer cover, the second machine-readable code including a second digital content link.

In some aspects, in addition, or in alternative to any preceding aspects, the second machine-readable code is different from the first machine-readable code and replaces the first machine-readable code when the reactive ink transitions from the first state to the second state, and the second digital content link is different from the first digital content link.

In some aspects, in addition, or in alternative to any preceding aspects, the first machine-readable code and the second machine-readable code are 2-dimensional codes.

In some aspects, in addition, or in alternative to any preceding aspects, the first machine-readable code and the second machine-readable code are Quick Response (QR) codes.

In some aspects, in addition, or in alternative to any preceding aspects, the first digital content link and the second digital content link are uniform resource locators (URLs).

In some aspects, in addition, or in alternative to any preceding aspects, scanning the first machine-readable code with a user device causes the user device to automatically access, via the first digital content link, first digital content and display the first digital content to a user, and scanning the second machine-readable code with the user device causes the user device to automatically access, via the second digital content link, second digital content and display the second digital content to the user, the second digital content being different from the first digital content.

In some aspects, in addition, or in alternative to any preceding aspects, each of the first digital content link and the second digital content link can be accessed by a computing device to present, to a user, at least one of written or graphical content, a video, an augmented reality (AR) experience, or a virtual reality (VR) experience.

In some aspects, in addition, or in alternative to any preceding aspects, the at least one of written or graphical content, a video, an AR experience, or a VR experience are presented via a user interface of the computing device.

In some aspects, in addition, or in alternative to any preceding aspects, the absorbent article is one of a diaper, a toilet training pant, an adult incontinence product, or a feminine hygiene product.

In some aspects, in addition, or in alternative to any preceding aspects, the activation event is contact with a bodily fluid.

In some aspects, in addition, or in alternative to any preceding aspects, the activation event is contact with a urine.

In some aspects, in addition, or in alternative to any preceding aspects, the activation event is one of change in temperature, exposure to light, exposure to a gas, or exposure to a microbial load.

Some of the aspects of the present disclosure relate to a method that includes providing an absorbent article configured to be worn by a person, the absorbent article including a reactive graphic visible at a first position on an exterior of the absorbent article, the reactive graphic including a reactive ink that, when exposed to an activation event, transitions from a first state to a second state.

In some aspects, in addition, or in alternative to any preceding aspects relating to the method, the reactive graphic forms a first machine-readable code at the first position on the exterior of the absorbent article in the first state and a second machine-readable code at the first position on the exterior of the absorbent article in the second state, and the second machine-readable code is different from, and replaces, the first machine-readable code.

In some aspects, in addition, or in alternative to any preceding aspects relating to the method, the method further includes detecting, using a camera embedded within a computing device, the first machine-readable code, presenting, via the computing device, first digital content based on the first machine-readable code, and after the reactive ink has transitioned from the first state to the second state, detecting, using the camera embedded within a computing device, the second machine-readable code, and presenting, by the computing device, second digital content based on the second machine-readable code, and the second digital content is different from the first digital content.

In some aspects, in addition, or in alternative to any preceding aspects relating to the method, the first machine-readable code and the second machine-readable code are Quick Response (QR) codes.

In some aspects, in addition, or in alternative to any preceding aspects relating to the method, each of the first machine-readable code and the second machine-readable code are encoded with a unique web address that, when obtained by the computing device, cause the computing device to automatically access, via their respective unique web addresses, the first digital content or the second digital content.

In some aspects, in addition, or in alternative to any preceding aspects relating to the method, each of the first digital content and the second digital content comprise at least one of written or graphical content, a video, an augmented reality (AR) experience, or a virtual reality (VR) experience.

In some aspects, in addition, or in alternative to any preceding aspects relating to the method, the first digital content and the second digital content are presented via a user interface of the computing device.

In some aspects, in addition, or in alternative to any preceding aspects relating to the method, the first machine-readable code and the second machine-readable code are detected by scanning, using the camera of the computing device, the reactive graphic of the absorbent article without storing any captured images or video.

In some aspects, in addition, or in alternative to any preceding aspects relating to the method, the method further includes transmitting, to a remote computing device, data for one or both of the first digital content and the second digital content, which causes the remote computing device to present the first digital content or the second digital content.

In some aspects, in addition, or in alternative to any preceding aspects relating to the method, the absorbent article is one of a diaper, a toilet training pant, an adult incontinence product, or a feminine hygiene product.

In some aspects, in addition, or in alternative to any preceding aspects relating to the method, the activation event is one of exposure to a bodily fluid, change in temperature, exposure to light, exposure to a gas, or exposure to a microbial load.

Additional aspects of the disclosure will be set forth, in part, in the detailed description, figures, and claims which follow, and in part will be derived from the detailed description or can be learned by practice of the disclosure. It is to be understood that both the foregoing general description and the following detailed description are exemplary and explanatory only and are not restrictive of the disclosure as disclosed.

### BRIEF DESCRIPTION OF THE DRAWINGS

FIG. 1 is a plan view of an example absorbent article having a reactive graphic disposed thereon according to some implementations.

FIG. 2 is a diagram illustrating a transition of the reactive graphic from a first state to a second state according to some implementations.

FIG. 3 is a diagram of a system for scanning and processing a reactive graphic on an absorbent article according to some implementations.

FIG. 4 is a detailed block diagram of an example user device used in the system of FIG. 3 according to some implementations.

FIG. 5 is a flow diagram of a process for presenting unique digital content to a user based on a reactive graphic on an absorbent article according to some implementations.

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED DESCRIPTION

Reference now will be made in detail to various implementations of the disclosure, one or more examples of which are set forth below. Each example is provided by way of explanation and not limitation. In view of the disclosure, it will be apparent to those skilled in the art that various modifications and variations may be made to the various implementations described herein without departing from the scope or spirit of the disclosure.

As used herein, the term “absorbent article” refers to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, diaper pants, training pants, youth pants, swim pants, feminine hygiene products (including, but not limited to, menstrual pads or pants), incontinence products and other adult care garments, medical garments, surgical pads and bandages, other personal care or health care garments, and the like without departing from the scope of the present disclosure.

As used herein, the term “reactive graphic” refers generally to an image, pattern, text, or the like formed, at least in part, by an ink, substance, or other material that changes in response to a trigger or activation event.

Various implementations of the present disclosure are directed to an absorbent article that includes a reactive graphic. In various implementations, the reactive graphic is configured to change in response to a trigger or activation event (e.g., in response to the presence of moisture or high temperature). For example, in certain implementations, the reactive graphic may appear initially in a first state, in which the reactive graphic defines a first machine-readable code (e.g., a QR code) comprising a first digital content link (e.g., a link to a website, application, image, virtual reality experience, or other digital content accessible by a user device, such as a smart phone or computer). When the reactive graphic is exposed to an activation event (e.g., exposure to a bodily fluid, change in temperature, etc.), the reactive graphic transitions from the first state into a second state. In the second state, the reactive graphic defines a second machine-readable code comprising a second digital content link, the second machine-readable code being unique from the first machine-readable code and the second digital content link being unique from the first digital content link. In various implementations, the second machine-readable code may entirely replace the first machine-readable code, or the second machine-readable code may result from only a portion of the first machine-readable code changing.

In various implementations, the reactive graphic may be printed on, embedded within, or otherwise incorporated into the absorbent article. In contrast to purely decorative graphics, various implementations of the reactive graphic described herein are interactive and dynamic, providing users (e.g., a wearer of the absorbent article or caretaker of the wearer) with varying digital content when scanned with an appropriate device (e.g., a smart phone or computer). Accordingly, a system for detecting the aforementioned reactive graphic and presenting related digital content to a user is also disclosed herein. The system generally includes a user-operated computing device (e.g., a smartphone) that can scan the reactive graphic (e.g., by reading the first machine-readable code or second machine-readable code) and present the user with a unique digital content (e.g., digital content associated with the scanned machine-readable code).

In various implementations, the reactive graphic may be configured to provide enhanced user information. For example, in certain implementations, the reactive graphic may be configured to provide an enhanced user experience for toilet training. In particular, digital content provided in response to user scanning of the reactive graphic can be configured for tracking toilet training progress, providing celebratory experiences when toilet training goals are met, and providing encouraging content for toilet training children.

Accordingly, the type of digital experience presented to a user may be dependent on a state of the reactive graphic. For example, if the reactive graphic reacts to bodily fluids, then different digital content can be presented when the absorbent article (e.g., training pants) is dry versus wet. It should also be appreciated that the reactive graphic and related system described herein can be configured for a variety of absorbent article use cases, such as medical and adult care, childcare, and the like. Additional features of the absorbent material including a reactive graphic and related system are described in detail below.

Absorbent Article with Reactive Graphic

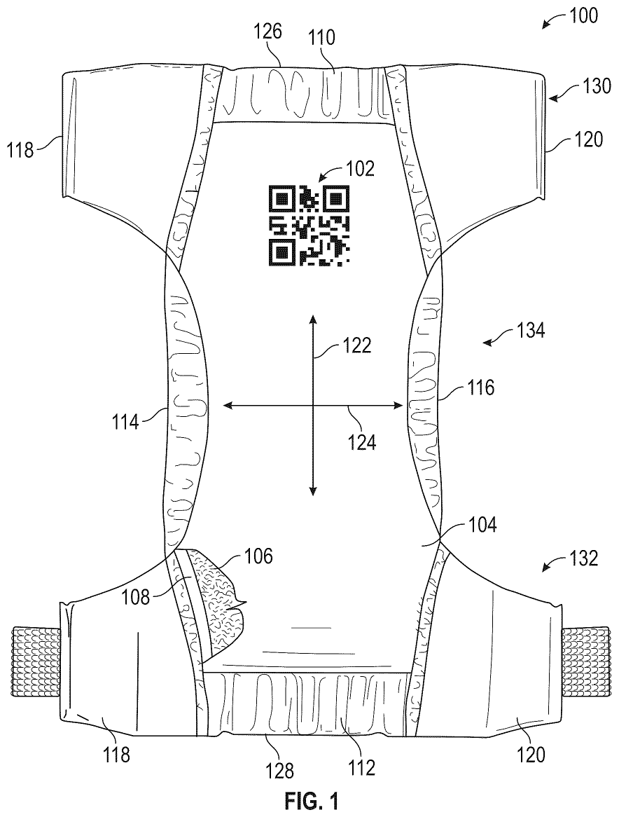

Referring first to FIG. 1, a diagram of an example absorbent article 100 having a reactive graphic 102 disposed thereon is shown, according to some implementations. In FIG. 1, the absorbent article 100 is depicted as a child's training pant. It will be appreciated, however, that the absorbent article 100 can be any absorbent article. The absorbent article 100 includes an outer cover 104 defining an exterior surface and an interior surface. The absorbent article 100 also includes an absorbent structure 106, which is positioned adjacent the interior surface of the outer cover 104. In the illustrated implementation, the absorbent article 100 also includes a liquid permeable inner lining, referred to herein as a bodyside liner 108. As shown in FIG. 1, the absorbent structure 106 is positioned between the outer cover 104 and the bodyside liner 108. The absorbent article 100 also includes elastic waistbands 110 and 112, and elastic leg members 114 and 116 for securing the absorbent article to a user.

Generally, the absorbent article 100 can be made from various different materials. In some implementations, for example, the outer cover 104 may be made from a material that is substantially liquid impermeable, and can be elastic, stretchable or nonstretchable. In some implementations, the outer cover 104 can be a single layer of liquid impermeable material or may include a multi-layered laminate structure in which at least one of the layers is liquid impermeable. For instance, the outer cover 104 can include a liquid permeable outer layer and a liquid impermeable inner layer that are suitably joined together by a laminate adhesive. For example, in some implementations, the liquid permeable outer layer may be a spunbond polypropylene nonwoven web. The spunbond web may have, for instance, a basis weight of from about 15 gsm to about 25 gsm.

In various implementations, the inner layer of the outer cover 104 can be both liquid and vapor impermeable, or can be liquid impermeable and vapor permeable. The inner layer is suitably manufactured from a thin plastic film, although other flexible liquid impermeable materials may also be used. The inner layer generally prevents waste material from wetting articles such as bedsheets and clothing, as well as the wearer and caregiver. A suitable liquid impermeable film may be a polyethylene film having a thickness of about 0.2 mm. A suitable breathable material that may be used as the inner layer is a microporous polymer film or a nonwoven fabric that has been coated or otherwise treated to impart a desired level of liquid impermeability. Other “non-breathable” elastic films that may be used as the inner layer include films made from block copolymers, such as styrene-ethylene-butylene-styrene or styrene-isoprene-styrene block copolymers.

The bodyside liner 108 can be manufactured from a wide variety of web materials, such as synthetic fibers, natural fibers, a combination of natural and synthetic fibers, porous foams, reticulated foams, apertured plastic films, or the like. Various woven and nonwoven fabrics can be used for the bodyside liner 180. However, it will be appreciated that the bodyside liner 108 is generally compliant, soft feeling, and non-irritating to a wearer's skin. For example, the bodyside liner 108 can be made from a meltblown or spunbonded web of polyolefin fibers. In some implementations, the bodyside liner 108 can also be a bonded-carded web composed of natural and/or synthetic fibers. In some implementations, the bodyside liner 108 is a nonwoven bicomponent web having a basis weight of about 27 gsm. The nonwoven bicomponent can be a spunbond bicomponent web, or a bonded carded bicomponent web. Suitable bicomponent staple fibers include a polyethylene/polypropylene bicomponent fiber. In this particular implementation, the polypropylene forms the core and the polyethylene form the sheath of the fiber. Other fiber orientations, however, are possible.

The absorbent structure 106 generally includes one or more materials that are suitable for absorbing liquids. The absorbent structure 106 may be formed of, for example, cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof. In some implementations, the absorbent structure 106 is a matrix of cellulosic fluff and superabsorbent hydrogel-forming particles. The cellulosic fluff may comprise a blend of wood pulp fluff. One preferred type of fluff is a bleached, highly absorbent wood pulp containing primarily soft wood fibers.

In certain implementations, the absorbent structure 106 may include a superabsorbent material (e.g., present in the absorbent web) in an amount of from about 0 to about 90 weight percent based on total weight of the web. The web may have a density within the range of about 0.1 to about 0.45 grams per cubic centimeter. Superabsorbent materials are well known in the art and can be selected from natural, synthetic, and modified natural polymers and materials. For example, superabsorbent materials can be inorganic materials, such as silica gels, or organic compounds, such as crosslinked polymers. Typically, a superabsorbent material is capable of absorbing at least 15 times its weight in liquid, and suitably is capable of absorbing more than about 25 times its weight in liquid.

In addition to cellulosic fibers and superabsorbent materials, the absorbent structure 106 may also contain adhesive elements and/or synthetic fibers that provide stabilization and attachment when appropriately activated. Additives such as adhesives may be of the same or different aspect from the cellulosic fibers; for example, such additives may be fibrous, particulate, or in liquid form; adhesives may possess either a curable or a heat-set property. Such additives can enhance the integrity of the bulk absorbent structure, and alternatively or additionally may provide adherence between facing layers of the folded structure. The absorbent materials may be formed into a web structure by employing various conventional methods and techniques. For example, the absorbent web may be formed with a dry-forming technique, an airlaying technique, a carding technique, a meltblown or spunbond technique, a wet-forming technique, a foam-forming technique, or the like, as well as combinations thereof. Layered and/or laminated structures may also be suitable.

In some implementations, the absorbent web material that forms at least a part of the absorbent structure 106 may be a coform material. The term “coform material” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and superabsorbent particles or fibers, inorganic absorbent materials, treated polymeric staple fibers and the like. Any of a variety of synthetic polymers may be utilized as the melt-spun component of the coform material. For instance, in some implementations, thermoplastic polymers can be utilized. Some examples of suitable thermoplastics that can be utilized include polyolefins, such as polyethylene, polypropylene, polybutylene and the like; polyamides; and polyesters. In one embodiment, the thermoplastic polymer is polypropylene.

It is also contemplated that elastomeric absorbent web structures may be used. For example, an elastomeric coform absorbent structure having from about 35% to about 65% by weight of a wettable staple fiber, and greater than about 35% to about 65% by weight of an elastomeric thermoplastic fiber may be used to define absorbent pad structures according to the invention. As another example, a suitable absorbent elastic nonwoven material may include a matrix of thermoplastic elastomeric nonwoven filaments present in an amount of about 3 to less than about 20% by weight of the material, with the matrix including a plurality of absorbent fibers and a super-absorbent material each constituting about 20-77% by weight of the material. Absorbent elastic nonwoven materials are useful in a wide variety of personal care articles where softness and conformability, as well as absorbency and elasticity, are important. The absorbent web may also be a nonwoven web comprising synthetic fibers. The web may include additional natural fibers and/or superabsorbent material. The web may have a density in the range of about 0.1 to about 0.45 grams per cubic centimeter. The absorbent web can alternatively be a foam.

As shown in FIG. 1, the absorbent article 100 also includes side panels 118 and 120. In some implementations, the side panels 118 and 120 can be permanently bonded together or can be releasably attached to one another. In the example shown, for instance, the side panels 118 and 120 are shown in an unattached state. In general, the side panels 118 and 120 are made from an elastic material, such as an elastic laminate. As also shown in FIG. 1, the absorbent article 100 defines a longitudinal center line 122, a transverse center line 124, a first or front longitudinal end edge 126, and a second or back longitudinal end edge 128. The longitudinal axes lie in the plane of the article and is generally parallel to a vertical plane that bisects a standing wearer into left and right body halves when the article is worn. The transverse axes lie in the plane of the article generally perpendicular to the longitudinal axis.

In general, the absorbent article 100 can be divided, along the longitudinal axis, into a front region 130, a back region 132, and a crotch region 134 positioned in between front and back regions 130, 132. In some implementations, front region 130, back region 132, and crotch region 134 can all have approximately the same length in the longitudinal direction. Additionally, the absorbent article 100 can be divided into a front half and a back half. The front half, for instance, may extend in the longitudinal direction from the front edge to the midpoint of the product, while the back half may extend from the back edge to the midpoint of the product.

Still referring to FIG. 1, the reactive graphic 102 is shown to be visible on an exterior surface of absorbent article 100. In some implementations, the reactive graphic 102 is printed on an exterior surface of absorbent article 100. For example, the reactive graphic 102 may be printed on the outer cover 104. In some implementations, the reactive graphic 102 is printed on or otherwise embedded on another layer of absorbent article 100 such that it is still visible from the exterior of absorbent article 100. For example, the reactive graphic 102 may be embedded into an additional substrate (not shown) that is positioned between the outer cover 104 and absorbent structure 106. It will also be appreciated that the reactive graphic 102 may be applied so as to be visible from an inside surface of the article. For instance, when applied to a feminine hygiene product, the reactive graphic 102 may more appropriately be placed to be visible from the inside surface which is adjacent to the body of the wearer. In order to be visible from the interior surface, the active graphics may be applied to a liquid permeable bodyside liner, to a surge layer, to a portion of the absorbent core, or even to the outer cover material in certain implementations.

Generally, the reactive graphic 102 is any graphic that changes in appearance responsive to a trigger, such as exposure to an activation event. More specifically, the reactive graphic 102 may be formed of an ink, substance, or other material that reacts or otherwise changes (e.g., by appearing, disappearing, changing color or shade, etc.) in response to an activation event. In various implementations herein, the activation event is described as exposure to a bodily fluid (e.g., urine, sweat, saliva, blood, tears, etc.). However, it should be appreciated that the reactive graphic 102 can be configured to react in response to a variety of desired activation events, such as a change in temperature, or exposure to light, a gas, a microbial load (bacteria, fungi, viruses, or combination thereof), etc. Accordingly, the reactive graphic 102 may be formed of any suitable reactive ink(s) (or other suitably responsive substance or material) based on the intended activation event.

In some implementations, the reactive graphic 102 is formed of two or more different inks that each react differently to the activation event. For example, the reactive graphic 102 may be formed of a first ink that fades or disappears and a second ink that appears (or otherwise becomes visible) when exposed to the activation event. As another example, the inks used to create the reactive graphic 102 may each change to a different color when exposed to the activation event. In some implementations, the reactive graphic 102 is partially formed of a non-reactive, or permanent, ink that does not react to the activation event. For example, a first portion of the reactive graphic 102 may be formed of a permanent ink and a second portion of the reactive graphic 102 may be formed of a reactive ink such that only the second portion of the reactive graphic 102 is modified responsive to the activation event.

In some implementations, at least a portion of the reactive ink that forms the reactive graphic 102 is a color changing active graphic composition. In some such implementations, the color changing active graphic composition may include a matrix-forming component, a colorant, a surfactant, and/or a pH adjuster. In some implementations, the matrix-forming component may be a water-insoluble, film-forming polymer or an ink base, such as a flexographic varnish having an organic solvent base. In some implementations, the colorant can be a pH indicator, such as a charged pH indicator, capable of changing color in response to the presence of a fluid. In some implementations, the surfactant may include a charged surfactant that attracts the colorant or a combination of charged surfactants that attract the colorant and a neutral surfactant. In some implementations, the pH adjuster may include a low molecular weight organic acid and a high molecular weight organic acid. The matrix-forming component may comprise, for instance, an (meth)acrylate/(meth)acrylamide copolymer, a polyurethane adhesive, methylcellulose, and/or copolymers of vinylpyrrolidone and dimethylaminopropyl methacrylamide.

In some implementations, a color-changing composition that is water-resistant and water-insoluble is used for at least a portion of the reactive graphic 102. The color-changing composition can form thin films of various patterns and shapes on a substrate upon drying and the films can generate color upon contact with an aqueous medium. In some implementations, the composition includes various components dissolved in a volatile organic solvent medium. The components can comprise a leuco dye or a combination of leuco dyes, an electron-withdrawing color developer or a combination of color developers that can form color complexes when associated with the leuco dye under the proper conditions, and a separator or combination of separators, which when dissolved in the system in an adequate quantity, can prevent the formation of the color complexes. In some implementations, the components can be contained within a polymeric encapsulation matrix. The encapsulating matrix can contain at least one kind of polymeric resin that can form a thin film on substrate surfaces with good adhesion. In addition to the above, the solution may also contain various other additives to adjust physical properties.

Leuco dyes are compounds can interconvert between two or more forms, the different forms having different colors. The forms are distinguished by the presence of one or more carbon-carbon, carbon-oxygen, carbon-nitrogen, or oxygen-nitrogen bonds that are present in one form but the other. By way of example, appropriately substituted polyarylmethane compounds undergoes a pH-dependent transition between a cationic compound having a color, and a colorless lactone:

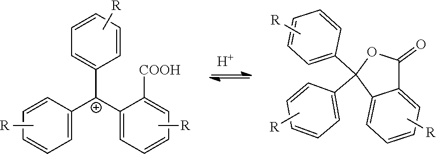

The prevalence of one form over another at any given pH may be controlled by selection of the R groups about the phenyl rings.

In some implementations, the leuco dye is a phthalide leuco dyes, arylmethane leuco dyes, fluoran leuco dyes, and combinations thereof.

In some implementations, the leuco dye is a triarylmethane-based dye, e.g. 3,3-bis(p-dimethylaminophenyl)-6-dimethylaminophthalide, 3,3-bis(p-dimethylaminophenyl) phthalide, 3-(p-dimethylaminophenyl)-3-(1,2-dimethylindol-3-yl) phthalide, 3-(p-dimethylaminophenyl)-3-(2-methylindol-3-yl) phthalide, 3,3-bis(1,2-dimethylindol-3-yl)-5-dimethylaminophthalide, 3,3-bis(1,2-dimethylindol-3-yl)-6-dimethylaminophthalide, 3,3-bis(9-ethylcarbazol-3-yl)-6-dimethylaminophthalide, 3,3-bis(2-phenylindol-3-yl)-6-dimethylaminophthalide, 3-p-dimethylaminophenyl-3-(1-methylpyrrol-3-yl)-6-dimethylaminophthalide, etc.

In some implementations, the leuco dye is a diarylmethane-based dye, e.g., 4,4′-bisdimethylaminobenzhydryl benzyl ether, N-halophenylleucoauramine, N-2,4,5-trichlorophenyl-leucoauramine, etc.

In some implementations, the leuco dye is a lactam-based dye, e.g., rhodamine-B-anilinolactam, rhodamine-(p-nitroanilino) lactam, rhodamine-(o-chloroanilino) lactam, etc.

In some implementations, the leuco dye is a fluoran-based dye, e.g., 3-dimethylamino-7-methoxyfluoran, 3-diethylamino-6-methoxyfluoran, 3-di-ethylamino-7-methoxyfluoran, 3-diethylamino-7-chlorofluoran, 3-diethylamino-6-methyl-7-chlorofluoran, 3-di-ethylamino-6,7-dimethylfluoran, 3-(N-ethyl-p-toluidino)-7-methylfluoran, 3-diethylamino-7-(N-acetyl-N-methylamino) fluoran, fluoran, 3-diethylamino-7-(N-methylamino) fluoran, 3-diethylamino-7-dibenzylaminofluoran, 3-diethylamino-7-(N-methyl-N-benzylamino) fluoran, 3-diethylamino-7-(N-chloroethyl-N-methylamino) fluoran, 3-diethylamino-7-N-diethylaminofluoran, 3-(N-ethyl-p-toluidino)-6-methyl-7-phenylaminofluoran, 3-(N-ethyl-p-toluidino)-6-methyl-7-(p-toluidino) fluoran, 3-diethylamino-6-methyl-7-phenylaminofluoran, 3-dibutylamino-6-methyl-7-phenylaminofluoran, 3-diethylamino-7-(2-carbomethoxyphenylamino) fluoran, 3-(N-cyclohexyl-N-methylamino)-6-methyl-7-phenylaminofluoran, 3-pyrrolidino-6-methyl-7-phenylaminofluoran, 3-piperidino-6-methyl-7-phenylaminofluoran, 3-diethylamino-6-methyl-7-(2,4-dimethylamino) fluoran, 3-diethylamino-7-(o-chlorophenylamino) fluoran, 3-dibutylamino-7-(o-chlorophenylamino) fluoran, 3-pyrrolidino-6-methyl-7-(p-butylphenylamino) fluoran, 3-(N-methyl-N-n-amylamino)-6-methyl-7-phenylaminofluoran, 3-(N-ethyl-N-n-amylamino)-6-methyl-7-phenylaminofluoran, 3-(N-ethyl-N-isoamylamino)-6-methyl-7-phenylaminofuluoran, 3-(N-methyl-N-n-hexylamino)-6-methyl-7-phenylaminofluoran, 3-(N-ethyl-N-n-hexylamino)-6-methyl-7-phenylaminofluoran, 3-(N-ethyl-N-β-ethylhexylamino)-6-methyl-7-phenylaminofluoran, etc.

The separator can be any of known component agent which exhibit good solubility in both water and organic solvents. In some implementations, the separators can be neutral molecules that are without a charge, such as polyalkylene glycols of <1000 Daltons, polyalkylene oxides of <10000 Daltons, block copolymers of polyoxyethylene polyoxypropylene glycol, polyoxyethylene nonylphenyl ether, polyoxyethylene distyrenated phenyl ether, and combinations thereof.

In some implementations, the separators can be glycerin; dodecylamine; 2,4,4-trimethyl-2-oxazoline; polyolefin glycols such as polyethylene glycol, polypropylene glycol and copolymers of ethylene glycol and propylene glycol; polyoxyethylene lauryl ether, polyoxyethylene oleyl ether, polyoxyethylene nonyl phenyl ether, polyoxyethylene sorbitan monolaurate, polyethylene glycol monostearate, and combinations thereof.

In some implementations, the activation event is exposure to a bodily fluid, especially urine. Urine typically occurs with a pH of 4.6 to 8, and healthy children excrete urine having a pH around 6. Certain medical conditions, however, are marked by abnormal pH levels in urine, and some implementations of the invention relate to diagnosis and/or detection of abnormal urine pH levels. For example, urinary tract infections can be characterized by elevated urine pH levels relative to healthy urine, and the activation event can include exposure to fluids having such elevated pH levels. As such provided herein are methods of detecting urinary tract infections using the compositions and articles disclosed herein. On the other hand, metabolic/respiratory acidosis can be characterized by lowered urine pH levels, and the activation event can include exposure to fluids having such decreased pH levels. As such provided herein are methods of detecting metabolic/respiratory acidosis (and related conditions) using the compositions and articles disclosed herein.

In certain implementations, the activation event is exposure to a fluid having a pH less than 9, less than 8.5, less than 8, less than 7.5, less than 7.0, less than 6.5, less than 6, less than 5.5, less than 5, less than 4.5 or less than 4. In certain implementations, the activation event is exposure to a fluid having a pH from 4-5.5, from 4.5-6, from 5-6.5, from 5-6, from 5-7, from 6-7, from 6-8, from 7-8, from 7-9, or from 8-9. In certain implementations the listed pH levels relate to urine. In some implementations the listed pH levels relate to saliva.

In some implementations, the reactive graphic 102 defines at least one machine-readable code that is at least partially formed (e.g., printed) with a reactive ink as described above. A machine-readable code is any graphic or symbol that stores information which can be obtained or accessed via scanning by a suitable computing device. Machine-readable codes can include, for example, 1-dimensional (1D) codes such as barcodes and/or 2-dimensional (2D) codes such as quick response (QR) codes. In the example of FIG. 1, the reactive graphic 102 includes a QR code, which is a 2D machine-readable code. A 2D code, for instance, can store information in multiple ways (e.g., in rows and columns or in a circular pattern). In some cases, 2D codes can use color in combination with a direction for storing information. In some implementations, a 2D code can include a matrix pattern that includes a finder component and an alignment component in addition to data components. In some implementations, 2D codes store text which may be immediately available once scanned with a suitable computing device. More commonly, however, 2D codes may include an embedded link (e.g., a URL) to a website or other remote resource. In some implementations, the link may be automatically accessed upon scanning on the 2D code. For example, scanning the QR code shown in FIG. 1 may cause the scanning device (e.g., a user's smartphone) to automatically navigate to a website that includes information related to absorbent article 100.

In some implementations, the reactive graphic 102 is incorporated into a scene, pattern, or other decorative graphics on absorbent article 100 in order to make the code less noticeable. For example, a scene may be printed on absorbent article 100 that includes various images, such as characters. A machine-readable code may be incorporated into the character or incorporated into the background of the scene. In some implementations, at least certain components of the scene are comprised of permanent graphics that surround the reactive graphic 102 that comprises the machine-readable code.

In some implementations, the reactive graphic 102 includes a first machine-readable code in a first or “initial” state. The first or “initial” state is generally prior to the reactive graphic 102 being exposed to an activation event. Upon exposure to an activation event (e.g., a bodily fluid, change in temperature, etc.), the reactive graphic 102 transitions from the first state to a second state, which is different from the first state. In some such implementations, this transition involves the machine-readable code transitioning from a first machine-readable code to a second machine-readable code (e.g., from a first QR code to a second QR code that is different from the first). For example, a first machine-readable code may be modified into a second machine-readable code, or the first machine-readable code may be completely replaced with a second machine-readable code.

In some implementations, the absorbent article 100 can further or alternatively include a material that reacts to an activation event (herein referred to as a “reactive material”). A reactive material is any material that changes at least one visual or measurable characteristic responsive to an activation event. For example, the texture, opaqueness, optical density, or any other characteristic of the reactive material may change when exposed to the activation event (e.g., heat, moisture, etc.). In some such implementations, the outer cover 104 of the absorbent article 100 may be at least partially formed of such a reactive material. For example, the entirety of the outer cover 104 may be formed of the reactive material, or a portion of the outer cover 104 (e.g., the portion containing the reactive graphic 102) may be formed of the reactive material. In some implementations, a reactive material is included in addition to the reactive graphic 102, such as to provide an indication of exposure to two different activation events. For example, the reactive graphic 102 may react to moisture and the reactive material to heat. In other implementations, a reactive material may be used in place of the reactive graphic 102.

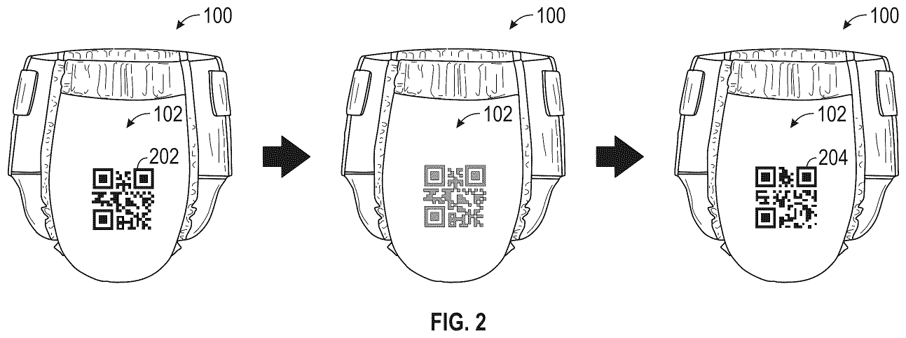

FIG. 2 illustrates an example of the reactive graphic 102 responding to an activation event according to some implementations. In particular, FIG. 2 shows the reactive graphic 102 transitioning from a first machine-readable code 202 to a second machine-readable code 204. In this example, first machine-readable code 202 is shown to gradually fade until it is replaced with second machine-readable code 204. For example, first machine-readable code 202 may be printed with a first ink that gradually lightens or disappears responsive to an activation event, while second machine-readable code 204 may be printed in a second ink that gradually darkens or appears responsive to the activation event. However, it should be appreciated that first machine-readable code 202 may be replaced by, or otherwise modified to be, second machine-readable code 204 in another of the other manners described above. For example, first machine-readable code 202 may be printed in both a reactive and non-reactive or permanent ink such that, upon exposure to the activation event, the reactive portions of first machine-readable code 202 change color, darken, or otherwise change visually to produce second machine-readable code 204. Notably, as shown in FIG. 2, second machine-readable code 204 is generally located at the same position or in the same region of absorbent article 100 as first machine-readable code 202. In other words, first machine-readable code 202 is replaced by second machine-readable code 204 or modified into second machine-readable code 204 within the region of the reactive graphic 102, which makes identification and locating of the reactive graphic 102 for scanning more user-friendly.

While reactive graphic 102 is generally described herein as being positioned on an absorbent article 100, it should be appreciated that, in various implementations, reactive graphic 102 could be incorporated into (e.g., printed on) other types of products. In some implementations, for example, reactive graphic 102 may be disposed on cleaning wipes. In some such implementations, the cleaning wipes may be disposable wipes, towels, or cloths that are used for general-purpose cleaning. In some such implementations, reactive graphic 102 may transition from the first state to the second state (e.g., from first machine-readable code 202 to second machine-readable code 204) when the cleaning wipe is exposed to, or contacted with, an activation agent such as a cleaning chemical (e.g., alcohol, degreaser, etc.). In other such implementations, the activation agent may be a particular contaminate on the surface or object being cleaned. In some implementations, a cleaning wipe may have multiple reactive graphics positioned thereon that each react to different activation agents.

### Digital Content Delivery

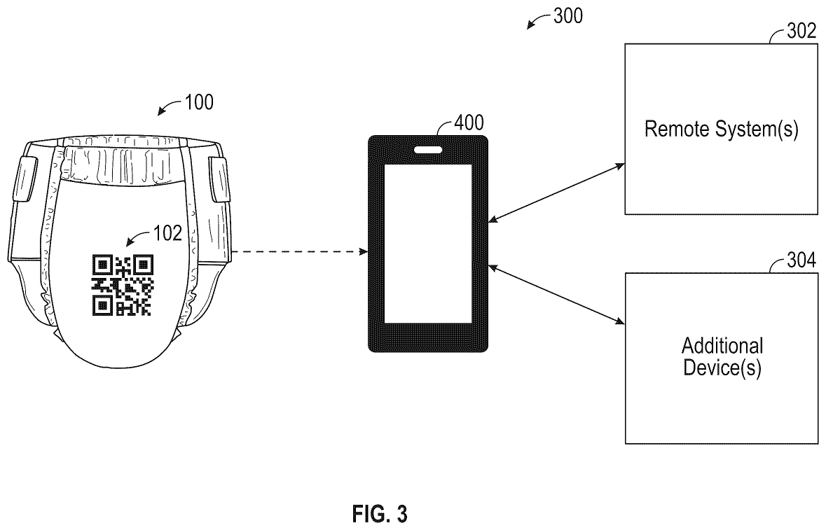

Referring now to FIG. 3, a diagram of a system 300 for scanning and processing a reactive graphic on an absorbent article is shown, according to some implementations. In this example, the reactive graphic is the reactive graphic 102, which includes a 2D machine-readable code and which is incorporated into (e.g., printed on) absorbent article 100. As shown, a suitable user device 400 may be used to scan the reactive graphic 102 in order to access or obtain the information embedded therein. In some implementations, the user device 400 is a handheld computing device that includes a camera or optical scanner. For example, the user device 400 may be a smartphone; however, it should be appreciated that the user device 400 may be any computing device that is capable of scanning a machine-readable code. For example, the user device 400 may be a desktop computer that includes a separate, handheld or mounted scanner for scanning machine-readable code. In other examples, the user device 400 may be an AR/VR headset, smart glasses, a security device (e.g., a security camera), or any other device that has a camera or image sensor capable of scanning machine readable codes. In some implementations, when the outer cover 104 of the absorbent article 100 is at least partially formed of a reactive material, the user device 400 is configured to detect changes in visual characteristics of the outer cover 104. For the sake of conciseness, such implementations are not described in detail herein; however, it will be appreciated that the following description of scanning the reactive graphic 102 is generally applicable to implementations where the user device 400 scans a reactive material.

Upon scanning the machine-readable code of the reactive graphic 102, the user device 400 may be configured to perform various operations based on the information embedded within the machine-readable code. For example, the user device 400 may simply display, via a user interface, text embedded within the machine-readable code. In some implementations, the user device 400 automatically accesses a link embedded within the machine-readable code. Alternatively, in some implementations, the user device 400 displays the extracted link to a user, thereby allowing the user to manually select the link to be presented with the associated digital content. As described above, for example, the machine-readable code may include a URL that can automatically be accessed to retrieve digital content (e.g., images, videos, etc.). In particular, following the embedded link within the machine-readable code may cause the user device 400 to access and present a digital user experience. “Digital content”, as described herein, generally refers to one or more images, videos, text, graphics, animations, music, and the like. In some cases, the digital content is interactive and may therefore be referred to herein as a “digital experience”. Accordingly, upon presenting a digital experience to a user, the user device 400 may receive user inputs related to the digital experience.

Notably, users of the user device 400 may be presented with at least two different sets or types of digital content based on the state of the reactive graphic 102 at the time of scanning. With reference to FIG. 2, for example, first digital content may be presented when first code 202 is scanned and second digital content may be presented when second machine-readable code 204 is scanned (e.g., after the reactive graphic 102 is exposed to an activation event). In this way, the digital content provided to the user is context-relevant and associated with the state of the reactive graphic 102 (and thereby the absorbent article 100).

In some implementations, the absorbent article comprises a child training pant which has the reactive graphic 102 arranged thereon. The reactive graphic 102 includes a first machine-readable code which reacts with body exudate (e.g., urine) to transition to a second machine-readable code. The first machine-readable code, which is visible prior to exposure to the body exudate (e.g., when the training pants are dry), can be scanned by the user device 400 to present the user with first digital content (e.g., a video, text and/or graphics, music, etc.). After the reactive graphic 102 is exposed to body exudate (e.g., when the training pants are wet), the first machine-readable code is replaced with the second machine-readable code, which can then be scanned by the user device 400 to present the user with a second, different experience (e.g., a different video, text and/or graphics, music, etc.).

In some implementations, the user device 400 obtains or accesses digital content by communicating with the remote system(s) 302. In some such implementations, the remote system(s) 302 may include a computing device (e.g., a server) that is connected to the Internet or another type of network such that the user device 400 can send data to and/or receive data from remote system(s). For example, the user device 400 may automatically navigate to a website or web service hosted by the remote system(s) 302 using a link embedded within the machine-readable code(s) of the reactive graphic 102. Put another way, the remote system(s) 302 may maintain and provide data relating to various digital content, which is then remotely accessed by the user device 400 in order to be presented to a user. In this manner, the remote system(s) 302 may handle at least part of the computational burden associated with presenting digital content.

In some implementations, the user device 400 can further communicate with additional device(s) 304 to present digital content. For example, the user device 400 may “cast” or share digital content with the additional device(s) 304. More specifically, in some implementations, the user device 400 may transmit digital content data to the additional device(s) 304 to cause the additional device(s) 304 to present the digital content. The additional device(s) 304 may include local computing devices and/or computing devices owned and/or operated by a user of the user device 400. For example, the additional device(s) 304 may include smart devices such as televisions, displays, speakers, tablets, additional smartphones, etc. In some implementations, the additional device(s) 304 can include computing devices that are capable of presenting virtual reality (VR) and/or augmented reality (AR) experiences to a user. For example, the additional device(s) 304 may include a VR/AR headset, glasses, or the like.

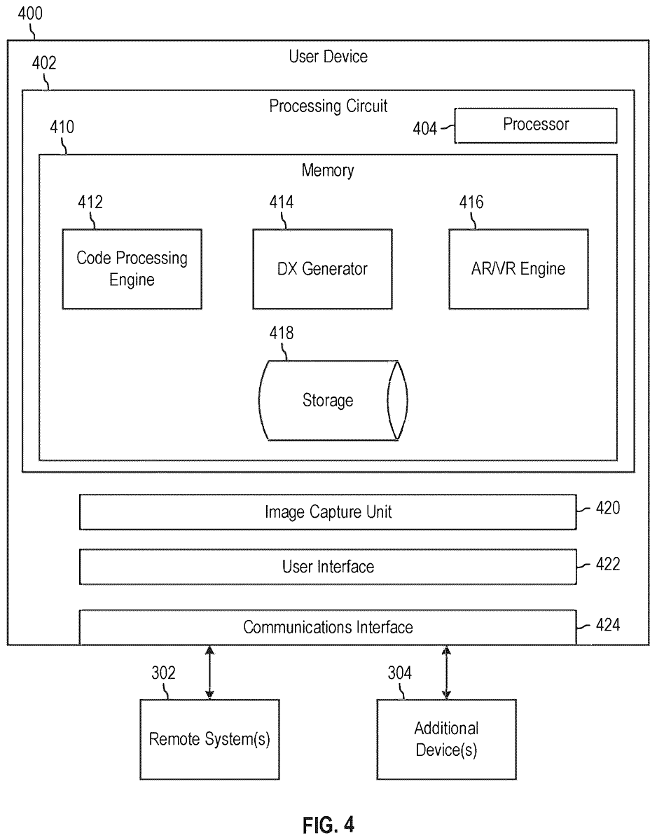

Referring now to FIG. 4, a detailed block diagram of the user device 400 is shown, according to some implementations. As mentioned above, the user device 400 is generally any computing device that is capable of scanning machine-readable codes, such as QR codes. In some implementations, the user device 400 is a handheld computing device, such as a smartphone or tablet. The user device 400 is shown to include a processing circuit 402 that includes a processor 404 and a memory 410. The processor 404 can be a general-purpose processor, an application specific integrated circuit (ASIC), one or more field programmable gate arrays (FPGAs), a group of processing components, or other suitable electronic processing structures. In some implementations, the processor 404 is configured to execute program code stored on memory 410 to cause the user device 400 to perform one or more operations, as described below in greater detail. In some implementations, the user device 400 is part of another computing device (not shown), such as a server. In some such embodiments, the components of the user device 400 described herein may be shared with, or the same as, the host device. It will also be appreciated that various functions of the user device 400 described herein may be implemented by other (e.g., remote) computing devices. For example, in some cases, the user device 400 is merely used to scan the reactive graphic 102, but the processing of the reactive graphic 102 is handled by another computing device.

Memory 410 can include one or more devices (e.g., memory units, memory devices, storage devices, etc.) for storing data and/or computer code for completing and/or facilitating the various processes described in the present disclosure. In some implementations, memory 410 includes tangible (e.g., non-transitory), computer-readable media that stores code or instructions executable by the processor 404. Tangible, computer-readable media refers to any physical media that is capable of providing data that causes the user device 400 to operate in a particular fashion. Example tangible, computer-readable media may include, but is not limited to, volatile media, non-volatile media, removable media and non-removable media implemented in any method or technology for storage of information such as computer readable instructions, data structures, program modules or other data. Accordingly, memory 410 can include RAM, ROM, hard drive storage, temporary storage, non-volatile memory, flash memory, optical memory, or any other suitable memory for storing software objects and/or computer instructions. Memory 410 can include database components, object code components, script components, or any other type of information structure for supporting the various activities and information structures described in the present disclosure. Memory 410 can be communicably connected to the processor 404, such as via the processing circuit 402, and can include computer code for executing (e.g., by the processor 404) one or more processes described herein.

While shown as individual components, it will be appreciated that the processor 404 and/or memory 410 can be implemented using a variety of different types and quantities of processors and memory. For example, the processor 404 may represent a single processing device or multiple processing devices. Similarly, memory 410 may represent a single memory device or multiple memory devices. Additionally, in some implementations, the user device 400 may be implemented within a single computing device (e.g., one server, one housing, etc.). In other implementations, the user device 400 may be distributed across multiple servers or computers (e.g., that can exist in distributed locations). For example, the user device 400 may include multiple distributed computing devices (e.g., multiple processors and/or memory devices) in communication with each other that collaborate to perform operations. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. For example, virtualization software may be employed by the user device 400 to provide the functionality of a number of computing devices.

Memory 410 is shown to include a code processing engine 412 which is configured to detect machine-readable codes and extract embedded information. In some implementations, to detect machine-readable codes, code processing engine 412 may continuously process image data from an image capture unit 420 of the user device 400. Image capture unit 420 may be any device or group of components that is capable of capturing image data and/or scanning machine-readable codes. For example, the image capture unit 420 may be a camera (e.g., a smartphone camera), a barcode scanner, an optical scanner, or the like. Accordingly, code processing engine 412 may continuously process image data captured by the image capture unit 420 to detect machine-readable codes and, once detected, to extract information from the machine-readable codes. In some implementations, code processing engine 412 utilizes artificial intelligence (AI) or machine learning (ML) to detect and extract information from machine-readable codes. However, it should be appreciated that code processing engine 412 may, more generally, use any suitable technique or algorithm for processing machine-readable codes. Typically, the information extracted for a machine-readable code (e.g., a QR code) is a string of alphanumeric characters. For example, a QR code (e.g., of the reactive graphic 102) may contain a string of characters that form a link (e.g., a URL) to an external resource (e.g., a website).

Memory 410 is further shown to include a digital experience (DX) generator 414 which generates and presents digital content and interactive experiences to a user. In some implementations, the DX generator 414 uses the information extracted from a machine-readable code, by the code processing engine 412, to generate said digital content. For example, responsive to the code processing engine 412 extracting a URL from a QR code, the DX generator 414 may be configured to automatically follow the URL to access a remote resource (e.g., a web page) and/or to retrieve additional information. In some implementations, digital content is as simple as text or images that can be presented to a user. However, the DX generator 414 may also be configured to obtain and present videos, music, and the like. In some implementations, the DX generator 414 may generate digital experiences (e.g., interactive digital content) using any form of digital media. In some implementations, rather than generating digital content or experiences directly, the DX generator 414 is configured to retrieve predefine or already-created digital content from the remote system(s) 302 and/or a database of digital content.

In some implementations, digital content is presented via a user interface 422 of the user device 400. User interface 422 generally includes a display (e.g., an LED or LCD display) and, in some implementations, may include a user input device (e.g., a mouse, a keyboard, a touchscreen, etc.). In some implementations, the user interface 422 further includes components such as a speaker or lights to provide additional outputs to a user. In some implementations, digital content is presented via an external device (e.g., one of the additional device(s) 304). In some such implementations, digital content may be presented via a television, a tablet, or another computing device that is remote from the user device 400. For example, the user device 400 may “cast” a digital experience to another device.

In some implementations, digital content can include AR/VR content (e.g., which may be considered a “digital experience”) or, put another way, can be presented using AR/VR. For example, a digital experience can be presented to a user that includes graphics, videos, text, etc., in an AR/VR environment. In some such implementations, memory 410 may include an AR/VR engine 416 that generates AR/VR content based on the obtained digital content data. For example, the AR/VR engine 416 may overlay a video feed (e.g., from the image capture unit 420) with additional digital content (e.g., graphics, videos, etc.) to generate an AR experience. In some implementations, AR/VR content is displayed via a separate device (e.g., one of the additional device(s) 304), such as a VR headset which is worn by a user. In some such implementations, the AR/VR engine 416 may generate a virtual environment populated with content relating to the digital content provided by the machine-readable code, which is then displayed via the separate device.

In some implementations, the DX generator 414 is further configured to receive user inputs based on presented digital content. For example, users may interact with presented content (e.g., selecting items from lists, entering text, speaking, etc.) and these user interactions may be detected and processed by the DX generator 414. In some implementations, user interactions and/or data from scanned machine-readable codes may be stored in a database, shown as storage 418. In some implementations, the storage 418 is used to track scanned codes to detect changes and provide additional feedback to a user. For example, information such as the time a code was scanned, a state of the code, whether the state of the code changed between scans, an amount of time between scans, etc., may be tracked, which can provide further insight to a user.

Consider, for example, the scenario described above relating to child's training pants. The user device 400 may be configured to record a state of the reactive graphic 102 responsive to an initial scan and may compare subsequent scans of the reactive graphic 102 to the initially recorded state, which may affect the digital content presented to a user. In the example of toilet training, a celebratory digital “experience” may be presented if the reactive graphic 102 is determined not to have changed states between scans (e.g., indicating that the wearer did not relieve themselves in the training pants). In this regard, it will be appreciated that reactive graphic 102 does not necessarily need to transition from the first state to the second state in order for a user to be presented with different or unique digital content. To continue the previous example, a toilet training child could be presented with a crown (e.g., a crown image, AR crown, or the like) the first time reactive graphic 102 is scanned without transitioning to the second state, and a crown and a scepter (e.g., a crown and scepter image, AR crown and scepter, or the like) the second time reactive graphic 102 is scanned without transitioning to the second state (e.g., indicating that the child maintained a dry absorbent article between scans of reactive graphic 102).

Still referring to FIG. 4, the user device 400 is also shown to include a communications interface 424 that facilitates communications between the user device 400 and any external components or devices, including the remote system(s) 302 and/or additional device(s) 304. Accordingly, the communications interface 424 can be or can include a wired or wireless communications interface (e.g., jacks, antennas, transmitters, receivers, transceivers, wire terminals, etc.) for conducting data communications, or a combination of wired and wireless communication interfaces. In some implementations, communications via the communications interface 424 are direct (e.g., local wired or wireless communications) or via a network (e.g., a WAN, the Internet, a cellular network, etc.). For example, the communications interface 424 may include one or more Ethernet ports for communicably coupling the user device 400 to a network (e.g., the Internet). In another example, the communications interface 424 can include a wireless (e.g., WiFi) transceiver for communicating via a wireless communications network. In yet another example, the communications interface 424 may include cellular or mobile phone communications transceivers and/or short-range transceivers (e.g., Bluetooth®).

While the user device 400 itself is generally capable of external communication for retrieving and/or presenting digital content, it will be noted that the bulk of the processing of machine-readable codes (e.g., detecting the codes, extracting information, etc.) may be performed locally by the various components of memory 410 to protect user privacy. For example, as described above, code processing engine 412 can detect machine-readable codes and extract data without communicating data (e.g., transmitting images) externally to the user device 400. In this manner, user privacy is protected. Additionally, in some implementations, code processing engine 412 is configured to prevent images from being captured while searching for machine-readable codes. As noted above, for example, the image capture unit 420 may be a camera that is integrated into the user device 400, and thus may be capable of capturing images; however, code processing engine 412 may prevent images from being captured (e.g., by disabling an image capture button) while searching for machine-readable codes to prevent saving images of absorbent article 100.

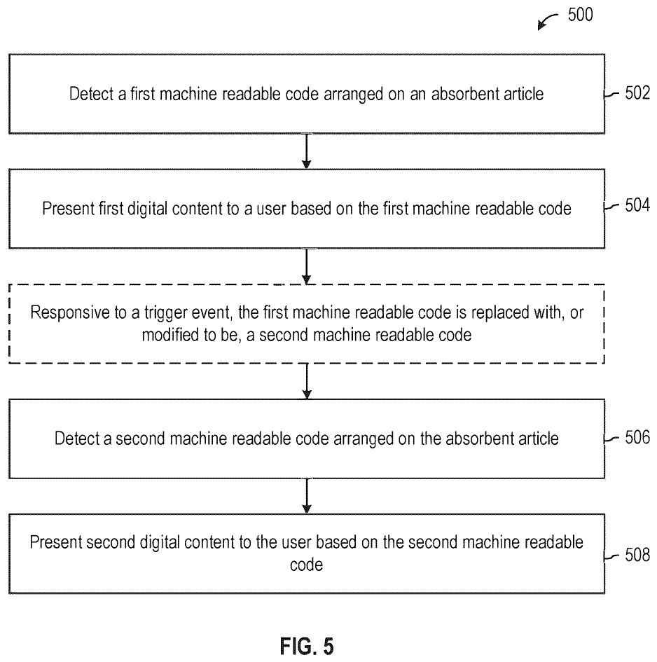

Referring now to FIG. 5, a flow diagram of a process 500 for presenting unique digital content to a user based on a reactive graphic on an absorbent article is shown, according to some implementations. In some implementations, process 500 is at least partially implemented by the user device 400, as described above. It will be appreciated that certain steps of process 500 may be optional and, in some implementations, process 500 may be implemented using less than all of the steps. It will also be appreciated that the order of steps shown in FIG. 5 is not intended to be limiting.

At step 502, a first machine-readable code arranged on an absorbent article is detected. In particular, the absorbent article may be absorbent article 100 which includes the reactive graphic 102, as described above. Accordingly, the first machine-readable code may correspond to the first or “initial” state of the reactive graphic 102, and thus is a 1D or 2D machine-readable code (e.g., a QR code) that is at least partially formed from a reactive ink that changes at least one visual characteristic responsive to an activation event. To this point, while not shown in FIG. 5, it should be appreciated that the absorbent article 100 having the reactive graphic 102 arranged thereon may be provided prior to initiating process 500. Notwithstanding, the first machine-readable code may be detected using any suitable computing device, such as the user device 400. For example, the user device 400 may be a smartphone with an integrated camera that is capable of detecting the first machine-readable code. In some implementations, the user device 400 is configured to continuously scan for machine-readable codes until the first machine-readable code is detected.

At step 504, first digital content is presented to a user based on the first machine-readable code. Upon detection, the user device 400 may be configured to automatically extract data from the first machine-readable code. As discussed above, the extract data may include a link (e.g., a URL) to a remote resource, such as a website, that the user device 400 automatically accesses to obtain the first digital content or data relating to the first digital content. This link may be referred to herein as a “digital content link.” The first digital content can include a variety of graphics, text, videos, animations, music, AR/VR content, and the like. As will be appreciated from the description herein, the first digital content may comprise digital content relevant to the first state of the reactive graphic 102 (e.g., digital content of interest to the user when an activation event has not occurred).

In some implementations, the first digital content is presented via a user interface of the user device 400. For example, AR content may be overlaid on a live video feed from a camera of the user device 400. As another example, a user interface may be displayed on the user device 400 that include text, graphics, etc. In some implementations, the first digital content is transmitted or “cast” to a secondary device, such as a television, a tablet, a display screen, or any other computing device. For example, the first digital content may be displayed via a television such that multiple users can view the experience. In another example, the first digital content is transmitted to an AR/VR capable device, such as a VR headset, for display.

As discussed herein, an activation event may occur which causes the reactive graphic 102 to transition from the first state to a second state. Generally, the second state is different from the first state. As described above, the trigger event may be exposure of the reactive graphic 102 to an activation event. For example, the activation event may be exposure to urine (in which case the reactive graphic 102 may transition to the second state in the presence of urine or other moisture), exposure to high temperatures (in which case the reactive graphic 102 may transition to the second state if a temperature threshold is exceeded), or exposure to bacteria and/or fungi (in which case the reactive graphic 102 may transition to the second state if the bacteria and/or fungi are present in/around absorbent article 100). In the second state, the reactive graphic 102 generally includes a second machine-readable code that is different from the first machine-readable code. As described above, for example, the second machine-readable code may completely replace the first machine-readable code, or the first machine-readable code may be modified into the second machine-readable code. In any case, at step 506, the second machine-readable code arranged on the absorbent article is detected in a similar manner as the first machine-readable code (e.g., by scanning with the user device 400).

At step 508, second digital content is presented to the user based on the second machine-readable code. As in step 504, the user device 400 may be configured to automatically extract data from the second machine-readable code, which may include a link (e.g., a URL) to a remote resource containing the second digital content (e.g., a second “digital content link”) or data relating to the second digital content. Like the first digital content, the second digital content may also include a variety of graphics, text, videos, animations, music, AR/VR content, and the like, and may be presented via a user interface of the user device 400 or another device. However, the second digital content is generally different from the first digital content. For example, the content and/or information presented in the second digital content may be different from the content and/or information presented in the first digital content. As will be appreciated from the description herein, the second digital content may comprise digital content relevant to the second state of the reactive graphic 102 (e.g., digital content of interest to the user when an activation event has occurred).

### USE CASE EXAMPLES

Absorbent article 100, and the related process 500 for presenting varying digital content based on a state of the reactive graphic 102 arranged thereon, may be useful in a variety of scenarios. For additional clarity, while example use cases are described herein, it should be appreciated that these examples are not intended to be limiting.

In implementation configured for toilet training (as described further herein), the user device 400 may be used to scan the reactive graphic 102 on the absorbent article 100 (e.g., toilet training pants) in order to present different digital content to a user (e.g., a parent) based on whether the training pants are wet or dry. If, for example, the training pants are dry, a celebratory experience may be presented (e.g., via the user device 400) to encourage the child. In this example, the user device 400 may track the state of the reactive graphic 102 at each scan to determine whether the wearer (e.g., a child) kept the training pants dry. In this regard, the reactive graphic 102 does not necessarily need to change (e.g., responsive to an activation event) to cause different digital content to be presented. Rather, different experiences may be presented based on tracked data. For example, different digital content may be presented when the reactive graphic 102 is scanned successive times while remaining the first state (e.g., presenting different encouraging content for potty training when the absorbent article 100 remains dry and the reactive graphic 102 is scanned in the first state on consecutive occasions). Furthermore, if the absorbent article 100 is wet—thus, the reactive graphic 102 has transitioned to the second state—a different digital experience may be presented that encourages the child to continue trying to keep the next absorbent article dry (e.g., presenting different encouraging content for potty training when the absorbent article 100 is wet and the reactive graphic 102 is scanned in the second state after previously being scanned in a first state). In this way, the digital content presented is associated with the state of the reactive graphic 102 (e.g., associated with the current state of the reactive graphic 102 and/or in relation to prior tracked states of the reactive graphic 102). In certain implementations, the digital content presented to the user may be randomized (e.g., by including a randomizer at the end of the first or second digital content links associated with the reactive graphic 102). In certain implementations, the user device and/or system may be configured to track progress against goals (e.g., tracking the number of successive times the reactive graphic 102 is scanned in the first state against a predefined goal, tracking the number of times any reactive graphic 102 is scanned in a second state over the course of a month against a predefined goal).

In another example, the reactive graphic 102 may be useful in tracking permanency. For example, the reactive graphic 102 may be formed with a reactive ink that changes responsive to temperature. In various implementations, the reactive graphic 102 may transition to the second state if the temperature of absorbent article 100 exceeds a temperature threshold. In such implementations, the reactive ink may be selected based on a desired temperature threshold. For example, in one embodiment, the reactive graphic 102 may transition from the first state to the second state if the reactive ink (forming the reactive graphic 102) is exposed to a temperature in excess of 100 degrees Fahrenheit. In another embodiment, the reactive graphic 102 may transition from the first state to the second state if the reactive ink (forming the reactive graphic 102) is exposed to a temperature in excess of 105 degrees Fahrenheit. Thus, if the threshold is exceeded, the reactive graphic 102 may maintain the second state (e.g., displaying a second QR code), which could signal to a parent or caretaker that the wearer of absorbent article 100 has been exposed to high or dangerous temperatures. For example, if the wearer of absorbent article 100 is a baby, this may indicate that the baby is over-dressed or otherwise at risk of a heat injury. In such implementations, the second machine-readable code (e.g., visible after the reactive graphic 102 is exposed to temperatures exceeding the temperature threshold) may be scanned to provide the user with instructions for care for children exposed to excessive heat, contact information for a medical provider, or the like.

In other implementations, the reactive graphic 102 may be formed with a reactive ink that responds to the presence of bacteria or fungus could indicate whether either are present in the wearer's body exude or in/around absorbent article 100. In this example, the second machine-readable code (e.g., visible after the reactive graphic 102 is exposed to the activation event) may be scanned to provide the user with instructions for care for children exposed to the bacteria or fungus for which the reactive graphic 102 is configured to respond to, contact information for a medical provider, or the like.

### Configuration of Certain Implementations

The construction and arrangement of the systems and methods as shown in the various implementations are illustrative only. Although only a few implementations have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

The present disclosure contemplates methods, systems, and program products on any machine-readable media for accomplishing various operations. The implementations of the present disclosure may be implemented using existing computer processors, or by a special purpose computer processor for an appropriate system, incorporated for this or another purpose, or by a hardwired system. Implementations within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

1. An absorbent article comprising:
an outer cover having an interior surface and an exterior surface;
an absorbent structure having a first surface positioned proximate to the interior surface of the outer cover and a second surface that is opposite the first surface and faces a wearer; and
a reactive graphic visible at a first position on the exterior surface of the outer cover, the reactive graphic comprising a reactive ink that, when exposed to an activation event, transitions from a first state to a second state;
wherein, in the first state, the reactive graphic forms a first machine-readable code at the first position on the exterior surface of the outer cover, the first machine-readable code comprising a first digital content link;
wherein, in the second state, the reactive graphic forms a second machine-readable code at the first position on the exterior surface of the outer cover, the second machine-readable code comprising a second digital content link;
wherein the second machine-readable code is different from the first machine-readable code and replaces the first machine-readable code when the reactive ink transitions from the first state to the second state, and wherein the second digital content link is different from the first digital content link.

2. The absorbent article of claim 1, wherein the first machine-readable code and the second machine-readable code are 2-dimensional codes.

3. The absorbent article of claim 2, wherein the first machine-readable code and the second machine-readable code are Quick Response (QR) codes.

4. The absorbent article of claim 1, wherein the first digital content link and the second digital content link are uniform resource locators (URLs).

5. The absorbent article of claim 1, wherein:
scanning the first machine-readable code with a user device causes the user device to automatically access, via the first digital content link, first digital content and display the first digital content to a user; and
scanning the second machine-readable code with the user device causes the user device to automatically access, via the second digital content link, second digital content and display the second digital content to the user, wherein the second digital content is different from the first digital content.

6. The absorbent article of claim 1, wherein each of the first digital content link and the second digital content link can be accessed by a computing device to present, to a user, at least one of written or graphical content, a video, an augmented reality (AR) experience, or a virtual reality (VR) experience.

7. The absorbent article of claim 6, wherein the at least one of written or graphical content, a video, an AR experience, or a VR experience are presented via a user interface of the computing device.

8. The absorbent article of claim 1, wherein the absorbent article is one of a diaper, a toilet training pant, an adult incontinence product, or a feminine hygiene product.

9. The absorbent article of claim 1, wherein the activation event is contact with a bodily fluid.

10. The absorbent article of claim 1, wherein the activation event is contact with a urine.

11. The absorbent article of claim 1, wherein the activation event is one of change in temperature, exposure to light, exposure to a gas, or exposure to a microbial load.

12. A method comprising:
providing an absorbent article configured to be worn by a person, the absorbent article comprising a reactive graphic visible at a first position on an exterior of the absorbent article, wherein the reactive graphic comprises a reactive ink that, when exposed to an activation event, transitions from a first state to a second state, wherein the reactive graphic forms a first machine-readable code at the first position on the exterior of the absorbent article in the first state and a second machine-readable code at the first position on the exterior of the absorbent article in the second state, wherein the second machine-readable code is different from, and replaces, the first machine-readable code;
detecting, using a camera embedded within a computing device, the first machine-readable code;
presenting, via the computing device, first digital content based on the first machine-readable code;
after the reactive ink has transitioned from the first state to the second state, detecting, using the camera embedded within a computing device, the second machine-readable code; and
presenting, by the computing device, second digital content based on the second machine-readable code, wherein the second digital content is different from the first digital content.

13. The method of claim 12, wherein the first machine-readable code and the second machine-readable code are Quick Response (QR) codes.

14. The method of claim 12, wherein each of the first machine-readable code and the second machine-readable code are encoded with a unique web address that, when obtained by the computing device, cause the computing device to automatically access, via their respective unique web addresses, the first digital content or the second digital content.

15. The method of claim 12, wherein each of the first digital content and the second digital content comprise at least one of written or graphical content, a video, an augmented reality (AR) experience, or a virtual reality (VR) experience.

16. The method of claim 12, wherein the first digital content and the second digital content are presented via a user interface of the computing device.

17. The method of claim 12, wherein the first machine-readable code and the second machine-readable code are detected by scanning, using the camera of the computing device, the reactive graphic of the absorbent article without storing any captured images or video.

18. The method of claim 12, further comprising transmitting, to a remote computing device, data for one or both of the first digital content and the second digital content, which causes the remote computing device to present the first digital content or the second digital content.

19. The method of claim 12, wherein the absorbent article is one of a diaper, a toilet training pant, an adult incontinence product, or a feminine hygiene product.

20. The method of claim 12, wherein the activation event is one of exposure to a bodily fluid, change in temperature, exposure to light, exposure to a gas, or exposure to a microbial load.
